SEQUENCE LISTING (8 sequences)

SEQ ID NO: 1 - protein, 12 aa, Artificial Sequence
  Specifically binds to the SARS-CoV-2 capsid protein
HISNHSHHHDIL

SEQ ID NO: 2 - protein, 12 aa, Artificial Sequence
  Specifically binds to the SARS-CoV-2 capsid protein
HMQSHKTHHSQR

SEQ ID NO: 3 - protein, 12 aa, Artificial Sequence
  Specifically binds to the SARS-CoV-2 capsid protein
HTVHAHHASHLS

SEQ ID NO: 4 - protein, 12 aa, Artificial Sequence
  Specifically binds to the SARS-CoV-2 capsid protein
IWGKSYHIHSLH

SEQ ID NO: 5 - protein, 7 aa, Artificial Sequence
  Specifically binds to the SARS-CoV-2 capsid protein
FSLPSTL

SEQ ID NO: 6 - protein, 8 aa, Artificial Sequence
  Specifically binds to the SARS-CoV-2 capsid protein
SFPVTLQK

SEQ ID NO: 7 - protein, 7 aa, Artificial Sequence
  Specifically binds to the SARS-CoV-2 capsid protein
TPIYHKL

SEQ ID NO: 8 - protein, 7 aa, Artificial Sequence
  Specifically binds to the SARS-CoV-2 capsid protein
HSMHHRH

(19) United States
(12) Patent Application Publication
(10) Pub. No.: US 2022/0365085 A1
(43) Pub. Date: Nov. 17, 2022

(54) BIORECEPTOR MOLECULES, THE USE OF BIORECEPTOR MOLECULES, SENSORS CONTAINING ELECTRODES MODIFIED WITH THE SAID BIORECEPTOR MOLECULES AND THE DETECTION METHOD OF SARS-COV-2 VIRUS

(71) Applicant: SENSDX SPÓLKA AKCYJNA, Warszawa (PL)

(72) Inventors: Wioleta BIALOBRZESKA, Gdansk (PL); Daniel BIGUS, Gdansk (PL); Zofia CEBULA, Gdansk (PL); Izabela ZALUSKA, Gdansk (PL); Joanna KRECZKO-KURZAWA, Gdansk (PL); Marceli MAKARUK, Gdynia (PL); Malgorzata BIEDULSKA, Banino (PL); Marta SOSNOWSKA, Pruszcz Gdanski (PL); Katarzyna PALA, Wroclaw (PL); Dawid NIDZWORSKI, Gdansk (PL); Krzysztof URBANSKI, Bielawa (PL); Paulina JANICKA, Janowice Wielkie (PL); Tomasz LEGA, Skowarcz (PL); Yanina DASHKEVICH, Gdansk (PL); Kamil WÓJCIK, Czechowice-Dziedzice (PL); Grzegorz HENIG, Mieroszow (PL)

(73) Assignee: SENSDX SPÓLKA AKCYJNA, Warszawa (PL)

(21) Appl. No.: 17/641,342

(22) PCT Filed: Jul. 17, 2020
(86) PCT No.: PCT/IB2020/056746

§ 371 Date: Mar. 8, 2022

(30) Foreign Application Priority Data
Sep. 9, 2019 (PL) P.431093

(51) Int. Cl. G01N 33/569; G01N 33/543; C07K 14/705; G01N 27/02

(52) U.S. Cl. CPC G01N 33/56983; G01N 33/5438; C07K 14/705; G01N 27/026

(57) ABSTRACT

The subject of the invention is a bioreceptor molecule with the formula: R₁-alkyl-C(0)NH—R₂, wherein alkyl is linear or branched alkyl with 2 to 20 C atoms; R₁ is selected from a group comprising thiol group (—SH); disulfide bridge; —S(O)-alkyl, wherein alkyl is linear or branched and contains 1-3 C atoms; thioether, wherein thioether contains 1-3 C atoms; thioacid; thionyl group; R₂ is a peptide with a sequence selected from a group comprising SEQ ID NO 1-8. Another subject of the invention is the use of bioreceptor molecules according to the invention in electrochemical impedance spectroscopy for detecting the SARS-CoV-2 virus. The subject of the invention is also a sensor containing an electrode, whose surface is covered with a layer of metal, characterized in that this layer is modified by bioreceptor molecules according to the invention. Furthermore, the subject of the invention is the method of detecting the SARS-Cov-2 virus by means of electrochemical impedance spectroscopy, including the following steps: a. rinsing and drying of the sensor electrode covered with metal; b. modification of the sensor electrode surface with bioreceptor molecules; c. calibration of the measurement system; d. detection of SARS-Cov-2 virus in a sample by means of a measurement system by observation of impedance changes, characterized in that surface modification of the sensor electrode is carried out using bioreceptor molecules according to the invention, wherein the presence of the virus in the test sample is indicated by a change in impedance of at least 10% in absolute value against the baseline value.

The invention concerns bioreceptor molecules, the use of bioreceptor molecules in electrochemical impedance spectroscopy for detecting pathogenic viruses in samples, sensors containing electrodes modified with these bioreceptor molecules and the method of virus detection by means of a measurement system modified with bioreceptor molecules using electrochemical impedance spectroscopy.

The recent outbreak of the pandemic (COVID-19) caused by the SARS-CoV-2 infection from Wuhan, China, poses a serious threat to global public health. Until the year 2002, human coronaviruses were harmless pathogens causing benign respiratory infections. The situation changed with the emergence of the SARS-CoV virus, responsible for severe acute respiratory syndrome. Despite the epidemiological risk associated with the emergence of SARS-CoV at that time and the current epidemiological problems associated with the emergence of SARS-CoV-2, no rapid and portable tests have yet been developed to detect human coronaviruses, which could be a remedy in a rapidly developing epidemic.

Such a virus test can provide valuable information to individuals or organisations trying to stop an epidemic, both locally and globally. It must be applicable for use on a large number of cases in a prospective manner to decide when people can be infectious, so that their participation in meetings, activities and travel can take place with the lowest risk of spreading the disease.

The ideal test for detecting SARS-CoV-2 should not only be fast, sensitive and specific, but also inexpensive and technologically simple, thanks to which it will be available at the place of care even in small hospitals or communities in developing countries. No tests designed to detect SARS-CoV-2 in clinical samples have so far met all these criteria, and effective detection of coronavirus is extremely important in the age of the existing threat of another human coronavirus outbreak.

The gold standard for the diagnosis of pathogen infections, including coronavirus detection, is the Real-Time-PCR method which allows for precise detection of microorganisms in samples (for example Ruifu Yang et al.; Real-Time Polymerase Chain Reaction for Detecting SARS Coronavirus, Beijing, 2003; Emerg Infect Dis. 2004 February; 10(2): 311-316; Peiris J S. et al.; Early diagnosis of SARS coronavirus infection by real time RT-PCR; J Clin Virol. 2003 December; 28(3):233-8 and Larry J. Anderson et al.; Real-Time Reverse Transcription-Polymerase Chain Reaction Assay for SARS-associated Coronavirus; Emerg Infect Dis. 2004 February; 10(2): 311-316). The PCR method is highly sensitive and in some cases may be quantitative. It also has some disadvantages such as a high price and long measurement time. In addition, the method requires specialized equipment, a laboratory and qualified personnel to operate it. Another limitation is that the molecular method does not distinguish between dead and active virus genetic material and therefore can detect RNA fragments that remain in the body after the patients have recovered, thus, giving false positive results.

An alternative is the ELISA immunoenzymatic assay, which allows for the identification of selected proteins (for example; Cheng Cao et al.; Diagnosis of Severe Acute Respiratory Syndrome (SARS) by Detection of SARS Coronavirus Nucleocapsid Antibodies in an Antigen-Capturing Enzyme-Linked Immunosorbent Assay; J Clin Microbiol. 2003 December; 41(12): 5781-5782, Kwok-Yung Yuen et al.; Detection of Severe Acute Respiratory Syndrome (SARS) Coronavirus Nucleocapsid Protein in SARS Patients by Enzyme-Linked Immunosorbent Assay; J Clin Microbiol. 2004 July; 42(7): 2884-2889 and U.S. patent application Ser. No. 10/983,854).

Both methods are expensive, require access to a biological laboratory and qualified personnel to operate them, so there is still a search for fast, easy to use and cheap diagnostic methods.

Lateral Flow tests are also known. This is a method similar to Rapid Influenza Diagnostic Tests (RIDT). The advantage of this method is simplicity of use, low cost and low time of measurement. The disadvantages are low sensitivity, low specificity and the impossibility to detect the virus in the early stages of infection (Olsen S J et al., Challenges With New Rapid Influenza Diagnostic Tests. Pediatr Infect Dis J. 2014 January; 33(1): 117-118; Koul P A et al., Performance of rapid influenza diagnostic tests (QuickVue) for Influenza A and B Infection in India. Indian J Med Microbiol. 2015 February; 33(Suppl): 26-31). These tests are based on antibodies that most often detect the surface proteins of the virus, therefore are sometimes unspecific during mutations.

There are many reports of virus detection biosensors in the scientific literature. Most of them are based on antibodies as molecules that recognize the virus and use different physical and chemical methods to generate signals. Seo G et al. (ACS Nano 2020, 14, 4, 5135-5142) describe a sensor based on FET (field-effect transistor) to detect SARS-CoV-2 in clinical samples. Qiu G. et al. (ACS Nano 2020) described a biosensor based on two methods, PPT (plasmonic photothermal) and LSPR (localized surface plasmon resonance) for detecting virus nucleic acids in clinical samples. The disadvantage of these solutions is their high level of complexity and early stage of development. The process of implementing such solutions on the market is very long, and the cost of the final product is high.

Precise pathogen detection can also be carried out by means of Electrochemical Impedance Spectroscopy (EIS), which is based on impedimentary bio-sensors. When a target substance, such as e.g. a protein, binds to receptor molecules previously bound to the surface of the bimpedance biosensor electrodes, the impedance value of the sensor changes. The difference in impedance measured before and after the binding of the target substance to the receptor molecules allows to detect the presence of the target substance in the solution.

The principle of EIS operation consists in determining the impedance of an electrochemical sensor by applying a small (typically several to several dozen millivolts) sine wave voltage of a specified frequency (typically between 1 mHz and 1 MHz) to the sensor electrodes and measuring the current flowing through the circuit/system. Additionally, electrochemical sensors are polarized with a DC voltage typically ranging from a few to several hundred millivolts, the purpose of which is to reduce the non-linearity of electrochemical sensor characteristics or to create conditions necessary for the occurrence of chemical reactions crucial for sensor operation. The advantage of Electrochemical Impedance Spectroscopy is that it is not necessary to modify the test with additional markers (e.g. fluorescent, radioactive and other dyes), thanks to which the interaction on the electrode surface is directly detected, which in turn increases the sensitivity of the test.

The publication Nidzworski et al. Scientific Reports, vol. 7, article no.: 15707 (2017), describes how to detect the influenza virus on BDD electrodes by EIS. The electrodes were modified with antibodies, selected for the M1 protein, which is universal for influenza viruses. The method is based on the use of polyclonal antibodies. The method of electrode modification as such is multistep and complex, and the use of antibodies involves additional limitations, such as storing the test under appropriate conditions.

As standard, antibodies recognising selected biomarkers are used to detect pathogens. Another way is to use aptamers, fragments of nucleic acids or fragments of antibodies or peptides. (Chiriaco et al, (Lab Chip, 2013, 13, 730); Molecules. 2018 Jul. 10; 23(7)). Some solutions use whole phages to recognize analytes. Antibodies are now the most widely used in diagnostics, due to their high affinity to the selected targets and relatively easy selection. Despite their versatility, antibodies are not ideal, especially in the context of the new PoC (Point of Care) rapid diagnostic methods. They are large proteins, which are relatively expensive to produce, and their attachment to the diagnostic test base is multistep. Moreover, due to their structure, they are sensitive to external conditions, such as for instance high temperature.

As an alternative to antibodies, short peptide sequences can be used to recognise selected molecular targets. These molecules are suitable for use in such diagnostic methods in which the strength of binding to a molecular target is not crucial, but specificity towards selected molecules is what matters.

Wide use of peptides is limited by their small size. In this case, it is difficult to construct a molecule that will continue to be selective towards selected pathogens, even after attaching to the test base.

In the case of short sequences, it is the whole molecule, not its fragment (as is the case with antibody interactions) that interacts with the analyte, which constitutes a limitation if the interacting molecule is attached to the substrate and not dissolved in solution.

There are several examples showing the use of peptides in the construction of sensors. These molecules have many advantages, but also limitations. One of their disadvantages is their small size, which means that the whole sequence is involved in recognizing the epitopes. This is not a problem for reactions carried out in a solution, but in the case of sensor structures where the molecule is attached to the base, there may be a steric hindrance that will prevent interaction with the analyte. In standard sensor modification procedures with small molecules, monolithic layers are formed on the sensor surface (see e.g. Molecules. 2018 Jul. 10; 23(7), FIG. 6). The only surface capable of interacting with pathogens or other biomarkers is therefore the last amino acid in the environment of other, tightly packed ones. This is one of the reasons why more often antibodies, aptamers, proteins or other molecules are used, which after immobilization on the surface of electrodes, do not form a steric hindrance limiting the interaction. One of the methods to partially solve this problem is the application of a molecule separating specific peptides such as β-mercaptoethanol (Lab Chip, 2013, 13, 730), but this procedure is not universal for a wide range of molecules.

Previously, the present inventors developed a method of detecting coexisting bacterial and viral pathogens with the use of a measurement system modified by specific bioreceptor molecules using electrochemical impedance spectroscopy—patent application P.431093, which is the priority for present patent application. The present invention is directed to the use of electrochemical impedance spectroscopy using suitably modified electrodes for SARS-CoV-2 virus detection. The subject of the present invention is a bioreceptor molecule with the following formula:

R₁-alkyl-C(O)NH—R₂
wherein alkyl is linear or branched alkyl with 1 to 20 C atoms;
R₁ is selected from the group comprising thiol group (—SH); disulfide bridge; —S(O)-alkyl, wherein alkyl is linear or branched and contains 1-3 C atoms; thioether, where thioether contains 1-3 C atoms; thioacid; thionyl group;
R₂ is a peptide with a sequence selected from a group comprising SEQ ID NO 1-8.

Preferably, R₁ is selected from the thiol group, disulfide bridge, —S(O)-alkyl, wherein alkyl is linear or branched and contains 1-3 C atoms. More preferably, R₁ is selected from the group comprising thiol group, the disulfide bridge.

Another subject of the invention is the use of bioreceptor molecules according to the invention in electrochemical impedance spectroscopy for detecting the SARS-CoV-2 virus.

The subject of the invention is also a sensor containing an electrode, the surface of which is covered with a layer of metal, characterized in that this layer is modified by bioreceptor molecules according to the invention.

Preferably, the surface of the electrode is covered with a layer of silver, copper, platinum or chemical, galvanic or evaporated gold.

In addition, the subject of the invention is the method of detecting the SARS-Cov-2 virus by means of electrochemical impedance spectroscopy, including the following steps:
a. washing and drying the metal-coated sensor electrode,
b. modification of the sensor electrode surface with bioreceptor molecules,
c. calibration of the measurement system,
d. detection of SARS-Cov-2 virus in a sample using measurement system by observation of impedance changes, characterized in that a modification of the sensor electrode surface is carried out with the use of bioreceptor molecules according to the invention, wherein the presence of the virus in the tested sample is evidenced by a change in impedance by an absolute value of at least 10% in relation to the baseline value.

The obtained spectra recorded by the SensDx MOBI reader (PCT/IB2019/050935) as a function of impedance and frequency are further analysed by the SensDx software in order to obtain the resistance value related to the limitation of the amount of transported electric charges, so called RCT (Charge Transfer Resistance), the value of which is a practical approximation of the overall impedance spectrum of the electrode.

If the pathogen is present, the reactions on the electrodes are expressed as follows:

R_{CTi} is the measured R_{CT} value of the modified electrode measured in pure PBS buffer before detection of proteins (the so-called ‘incubated’ value),
R_{CTr} is the R_{CT} value of the modified electrode measured in contact with the analyte containing the selected pathogen (SARS-CoV-2).

In the terms above concerning R_{CT} ‘i’ refers to ‘incubation’.—i.e. impedance measurement of an electrode modified by a bioreceptor molecule.

The suffix ‘r’ means ‘reaction’.—i.e. measurement of the modified electrode interaction with a pathogen. The impedance change is then calculated as:

Δ%=ABS[(R_{CTr}−R_{CTi})/R_{CTi}], where ABS is an absolute value.

The positive result is indicated by the dependency: Δ %>10%

For the skilled in the art, it will be obvious that the use of molecules developed in such way in the electrochemical impedance spectroscopy allowed to obtain a diagnostic test, the cost of which is significantly lower compared to the Gold Standard (Real-Time-PCR). Such a diagnostic test is also precise, fast and easy to use, what will be clear from the embodiments below.

The advantageous features of the invention are illustrated by the following Figures, supplementing the information contained in the embodiments.

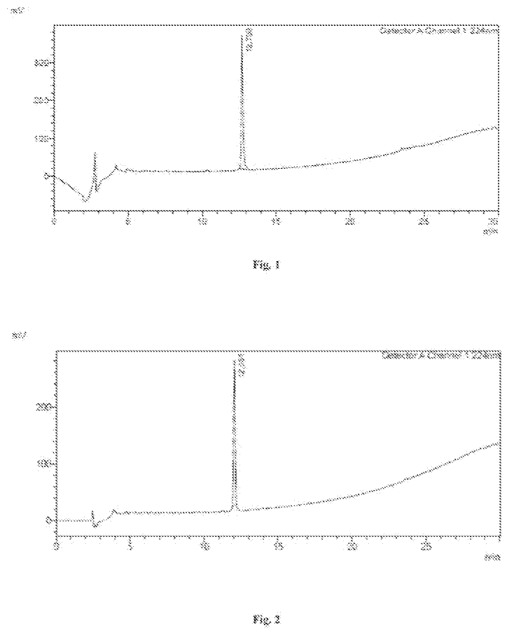

FIG. 1 shows the chromatogram of HPLC purification of 11-KOD1-NH2 molecule (SEQ ID NO 1)

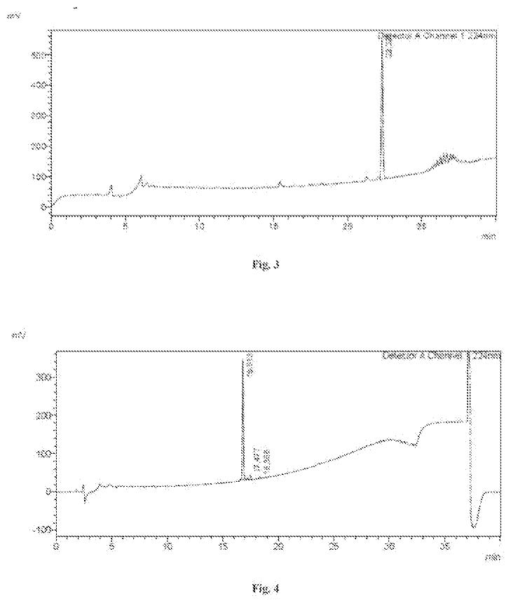

FIG. 2 shows the chromatogram of HPLC purification of 11-KOD2-NH2 molecule (SEQ ID NO 2)

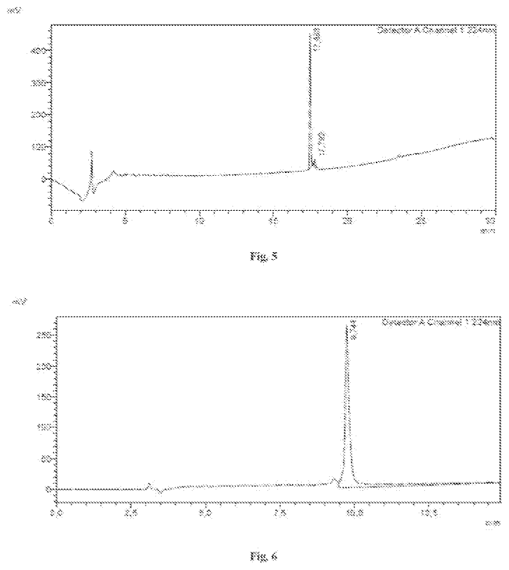

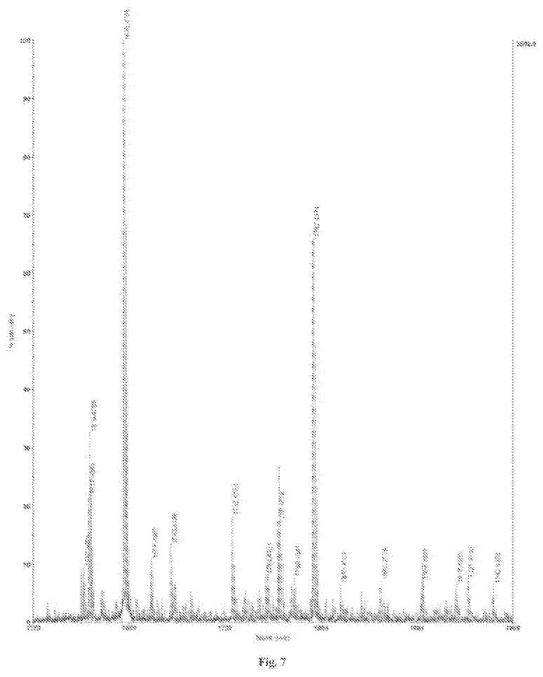

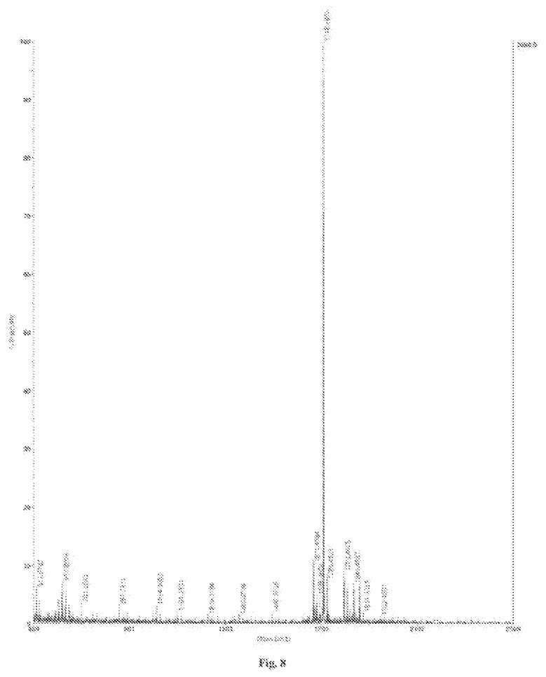

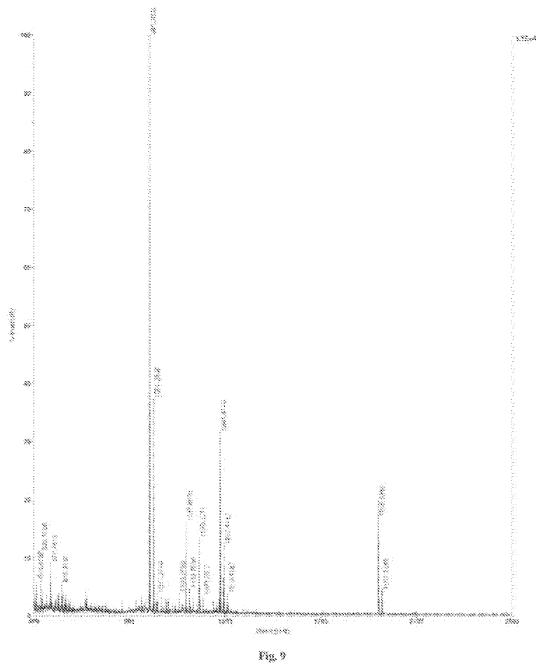

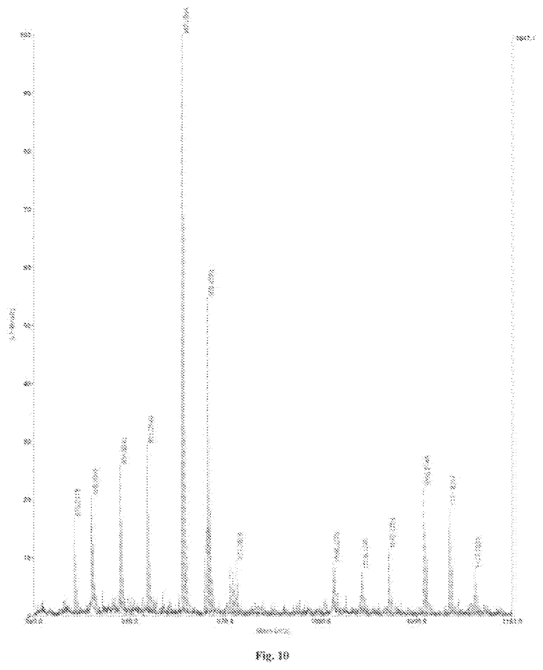

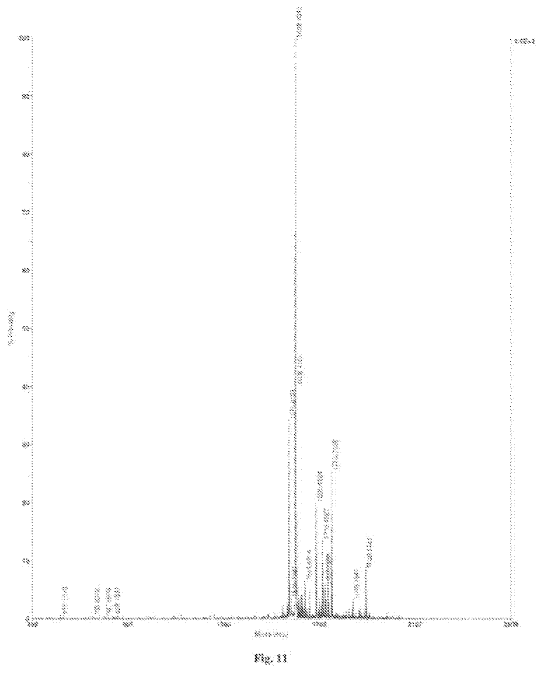

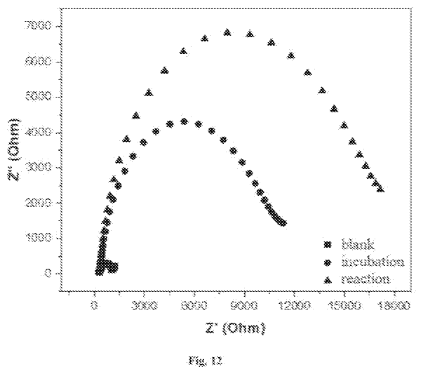

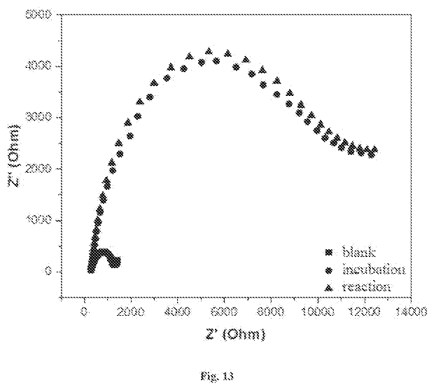

FIG. 3 shows the chromatogram of HPLC purification of 11-KOD5-NH2 molecule (SEQ ID NO 5)

FIG. 4 shows the chromatogram of HPLC purification of 11-KOD6-NH2 molecule (SEQ ID NO 6)

FIG. 5 shows the chromatogram of HPLC purification of 8-KOD5-NH2 molecule (SEQ ID NO 5)

FIG. 6 shows the chromatogram of HPLC purification of 8-KOD-1-NH2 molecule (SEQ ID NO 1)

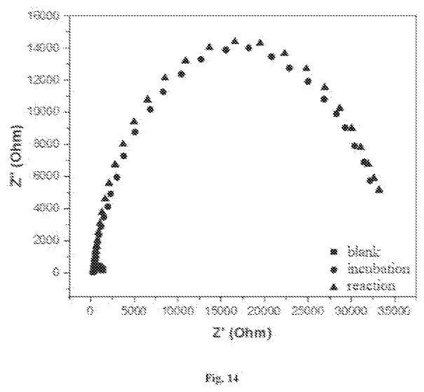

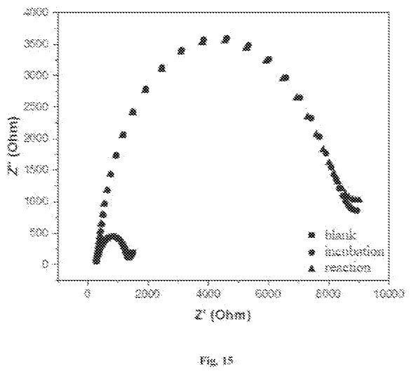

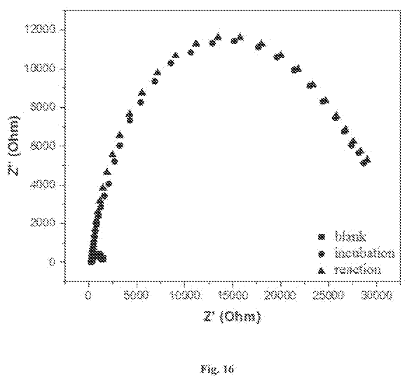

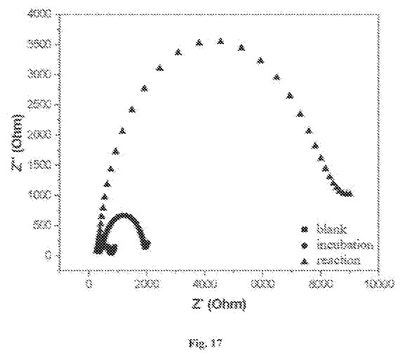

FIG. 7 shows the mass spectrometry spectrum for 11-KOD1-NH2 molecule

FIG. 8 shows the mass spectrometry spectrum for 11-KOD2-NH2 molecule

FIG. 9 shows the mass spectrometry spectrum for 11-KOD5-NH2 molecule

FIG. 10 shows the mass spectrometry spectrum for the 8-KOD5-NH2 molecule

FIG. 11 shows the mass spectrometry spectrum for the 8-KOD1-NH2 molecule

FIG. 12 shows the Nyquist diagram of the WHN-N protein interaction with the electrode modified with 11-KOD5-NH2 (SEQ ID NO 5). Blank—means the measurement of impedance on the unmodified electrode, incubation—measurement of impedance of the electrode modified with 11-KOD5-NH2, reaction—measurement of the modified electrode's interaction with the WHN-N protein.

FIG. 13 shows the Nyquist diagram of the Haemophilus influenzae bacteria interaction with the electrode modified with 11-KOD5-NH2. Blank—means the measurement of impedance on the unmodified electrode, incubation—measurement of impedance of the electrode modified with 11-KOD5-NH2 molecule, reaction—measurement of modified electrode's interaction with Haemophilus influenzae.

FIG. 14 shows the Nyquist diagram of the Streptococcus pyogenes bacteria interaction with the electrode modified with 11-KOD5-NH2 (SEQ ID NO 5). Blank—means the measurement of impedance on the unmodified electrode, incubation—measurement of impedance of the electrode modified with 11-KOD5-NH2 molecule, reaction—measurement of modified electrode's interaction with Streptococcus pyogenes.

FIG. 15 shows the Nyquist diagram of the Streptococcus pneumonia bacteria interaction with the electrode modified with 11-KOD5-NH2 (SEQ ID NO 5). Blank—means the measurement of impedance on the unmodified electrode, incubation—measurement of impedance of the electrode modified with 11-KOD5-NH2 molecule, reaction—measurement of modified electrode's interaction with Streptococcus pneumonia.

FIG. 16 shows the Nyquist diagram of the RSV virus interaction with the electrode modified with 11-KOD5-NH2 (SEQ ID NO 5). Blank—means the measurement of impedance on the unmodified electrode, incubation—measurement of impedance of the electrode modified with 11-KOD5-NH2 molecule, reaction—measurement of modified electrode's interaction with the RSV virus.

FIG. 17 shows the Nyquist diagram of the WHN-N protein virus interaction with the electrode modified with 11-KOD1-NH2 (SEQ ID NO 1). Blank—means the measurement of impedance on the unmodified electrode, incubation—measurement of impedance of the electrode modified with 11-KOD1-NH2 molecule, reaction—measurement of modified electrode's interaction with the WHN-N protein.

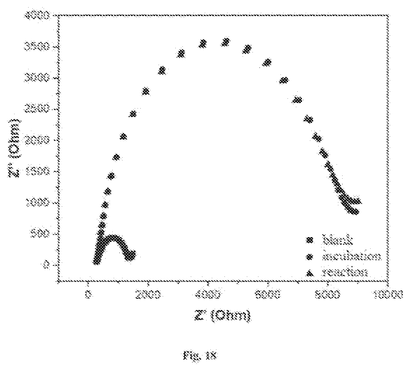

FIG. 18 shows the Nyquist diagram of the Haemophilus influenza bacteria interaction with the electrode modified with 11-KOD1-NH2 (SEQ ID NO 1). Blank—means the measurement of impedance on the unmodified electrode, incubation—measurement of impedance of the electrode modified with 11-KOD1-NH2 molecule, reaction—measurement of modified electrode's interaction with Haemophilus influenza.

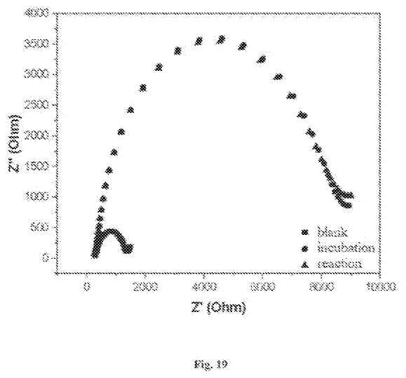

FIG. 19 shows the Nyquist diagram of the Streptococcus pyogenes bacteria interaction with the electrode modified with 11-KOD1-NH2 (SEQ ID NO 1). Blank—means the measurement of impedance on the unmodified electrode, incubation—measurement of impedance of the electrode modified with 11-KOD1-NH2 molecule, reaction—measurement of modified electrode's interaction with Streptococcus pyogenes.

FIG. 20 shows the Nyquist diagram of the Streptococcus pneumonia bacteria interaction with the electrode modified with 11-KOD1-NH2 (SEQ ID NO 1). Blank—means the measurement of impedance on the unmodified electrode, incubation—measurement of impedance of the electrode modified with 11-KOD1-NH2 molecule, reaction—measurement of modified electrode's interaction with Streptococcus pneumonia.

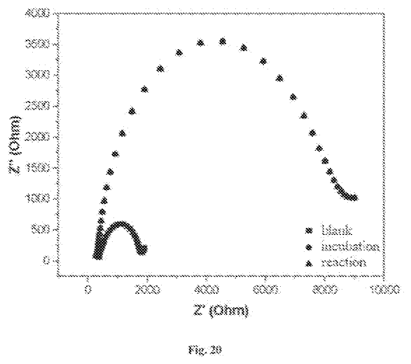

FIG. 21 shows the Nyquist diagram of the RSV virus interaction with the electrode modified with 11-KOD1-NH2. Blank—means the measurement of impedance on the unmodified electrode, incubation—measurement of impedance of the electrode modified with 11-KOD1-NH2 molecule, reaction—measurement of modified electrode's interaction with the RSV virus.

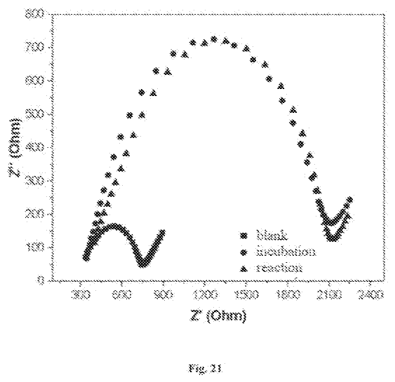

FIG. 22 shows the Nyquist diagram of the WHN-N protein interaction with the electrode modified with 11-KOD7-NH2 (SEQ ID NO 7). Blank—means the measurement of impedance on the unmodified electrode, incubation—measurement of impedance of the electrode modified with 11-KOD7-NH2 molecule, reaction—measurement of modified electrode's interaction with the WHN-N protein.

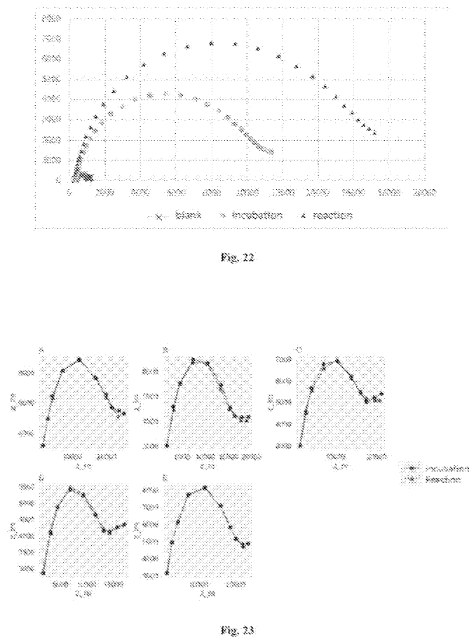

FIG. 23 shows the Nyquist diagram of Haemophilus influenzae (A), Streptococcus pneumoniae (B), Streptococcus pyogenes (C), RSV virus (D) and EBV (E) interaction with the electrode modified with 11-KOD7-NH2 (SEQ ID NO 7). Blank—means the measurement of impedance on the unmodified electrode, incubation—measurement of impedance of the electrode modified with 11-KOD7-NH2 molecule, reaction—measurement of modified electrode's interaction with the WHN-N protein.

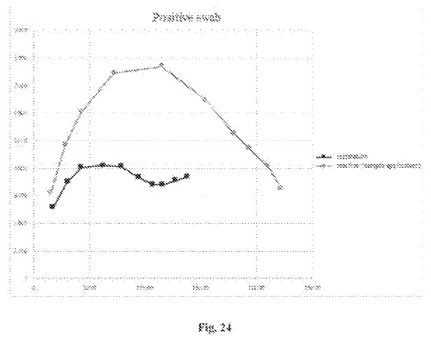

FIG. 24 shows the Nyquist diagram for a testing of a swab obtained from a patient infected with SARS-CoV-2, whose infection was confirmed by RT-PCR method, with a sensor modified with 11-KOD1-NH2 molecule (SEQ ID NO 1).

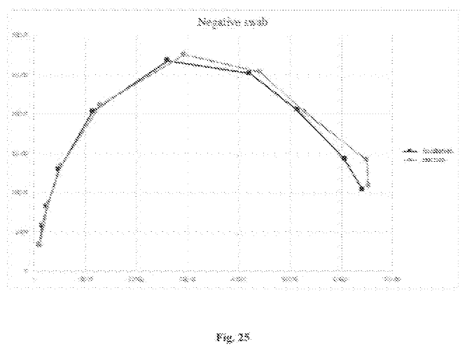

FIG. 25 shows the Nyquist diagram for a testing of a swab obtained from a patient not infected with SARS-CoV-2, whose absence of infection was confirmed by RT-PCR, with a sensor modified with 11-KOD1-NH2 molecule (SEQ ID NO 1).

FIG. 26 shows the cumulative results for a testing of a swab obtained from the positive patients infected with SARS-CoV-2 in which following modified sensors have been used: 8-COD1-NH2 (SEQ ID NO 1) (A), 11-KOD3-NH2 (SEQ ID NO 3) (B), 11-KOD4-NH2 (SEQ ID NO 4) (C), 8-KOD5-NH2 (SEQ ID NO 5) (D), 11-KOD6-NH2 (SEQ ID NO 6) (E) and 8-KOD7-NH2 (SEQ ID NO 7) (F).

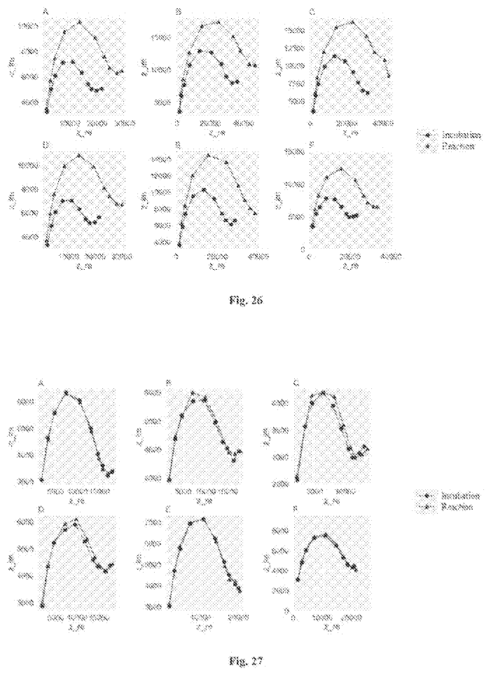

FIG. 27 shows the cumulative results for a testing of a swab obtained from the negative patients (not infected with SARS-CoV-2) in which following modified sensors have been used: 8-COD1-NH2 (SEQ ID NO 1) (A), 11-KOD3-NH2 (SEQ ID NO 3) (B), 11-KOD4-NH2 (SEQ ID NO 4) (C), 8-KOD5-NH2 (SEQ ID NO 5) (D), 11-KOD6-NH2 (SEQ ID NO 6) (E) and 8-KOD7-NH2 (SEQ ID NO 7) (F).

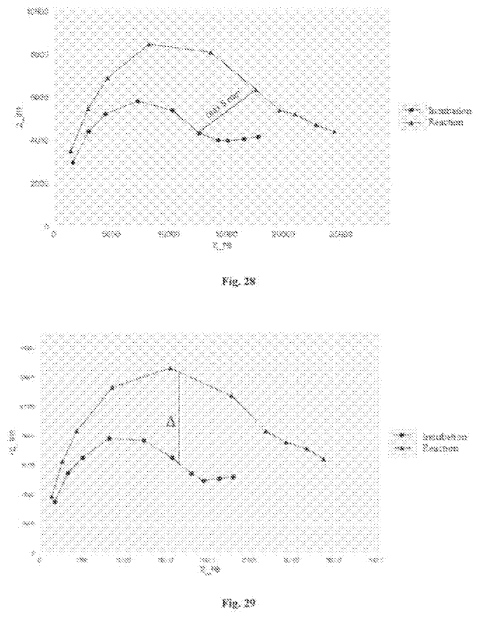

FIG. 28 shows the schematic time of positive swab measurement (max. 5 minutes from adding the sample to obtaining the result).

FIG. 29 shows a schematic result of the difference between the reference value (incubation) and the tested sample (reaction) indicating a positive result for SARS-CoV-2, i.e. an impedance change ‘Δ’ greater than 10%.

### EMBODIMENTS

### Example 1

### Selection Procedure for Peptide Sequences

For the selection of SARS-CoV-2 virus-specific binding sequences, the nucleocapsid N protein was selected, hereinafter referred to as WHN-N protein.

The peptide selection was carried out with the M13 phage library according to the standard procedure. 15 μg of WHN-N biomarker in TBS buffer was applied to microtiter plates and incubated at 4° C. overnight. Surfaces of wells were then blocked for 1 hour at 4° C. with 0.5% BSA diluted in TBS. Subsequently, approximately 1×10¹¹ phage forming units (pfu) were diluted in 100 μl TBS buffer with 0.1% TWEEN® 20 for 1 hour at room temperature with agitation. After incubation, wells were washed ten times with TBS buffer with 0.5% Tween-20. Bacteriophages were eluted with 0.2 M glycin-HCl, 0.1% BSA (pH 2.2) and amplified by host cell infection with E. coli ER2738. After 4.5 hours of growth at 37° C. the multiplied bacteriophages were separated from bacterial cells by centrifugion. The phages present in the supernatant were precipitated by addition of ⅙ volume of PEG/NaCl solution (20% w/v polyethylene glycol-8000; 2.5 M NaCl) and incubated for 16 hours at 4° C. The solution was centrifuged and the sediment was suspended again in 1 mL TBS buffer and titrated to determine the phage concentration. The procedure was repeated 3 times, after which the phages were plated and random plaques were selected. After amplification, the phage was purified by precipitation in PEG/NaCl and then suspended in 1/50 of the original volume in TBS buffer. Single-stranded DNA was isolated by incubation of bacteriophages in iodide buffer (4 M NaI, 1 mM EDTA in 10 mM Tris-HCl, pH 8.0) in order to denature the phage protein shell. The released DNA was then precipitated in 70% ethanol. The purified DNA was sequenced by the Genomed company (Poland).

### Example 2

### Synthesis and Purification of the Bioreceptor Molecule

Peptides were obtained using an automatic synthesizer with a pipetting arm, using the solid phase peptide synthesis (SPPS) method, using the Fmoc/tBu^{t} procedure. The syntheses were performed using Rink Amide AM resin (Deposition degree: 0.7 mmol/g). All reagents used had a high degree of purity (>95%, >97%, >98%, or analytical grade) and were purchased from the following manufacturers: Sigma Aldrich, VWR Chemicals, POCH S.A., P.P.H Stanlab, Iris Biotech GmbH, Alfa Aesar, Acros Organics, Thermo Fisher Scientific.

The synthesis was carried out using a module allowing for simultaneous synthesis of 8 independent peptide sequences with the use of disposable synthesis columns equipped with a sinter enabling drainage of the resin from the synthesis mixture.

Prior placing in the synthesizer, the resin was swelled for 30 minutes by cyclic rinsing 3×DMF, 3×DCM, 3×DMF. After that time the columns containing the resin were placed in the synthesizer in order to carry out the automatic synthesis cycles.

The automatic synthesis consisted of 7 to 12 (depending on the sequence) repeated steps of Fmoc protection group deprotection from α-amino group, rinsing and attachment of another protected amino acid derivative. During the deprotection step, Fmoc protection groups were removed with 20% piperidine solution in DMF.

In order to synthesize the indicated sequences, the 150 μmol scale method was used, with 4 times excess of acylating reagents. The reaction was carried out at 40° C. The acyclic mixture consisted of uniform amounts of Fmoc-AA: TBTU: HOBt: NMM dissolved in DMF.

A record of a single synthetic cycle is shown below:
Deprotection (2500 μl 20% piperidine in DMF) 8 min×1
Deprotection (2500 μl 20% piperidine in DMF) 12 min×1
Rinsing (2210 μl DMF) 1 min×4
Acylation (1560 μl of TBTU/HOBt mixture in DMF+390 μl NMM+10 μl NMP+1638 μl Fmoc-AA) 30 min×3
Rinsing (2210 μl DMF) 1 min×5

The last cycle of synthesis was followed by the final step of deprotection, rinsing and drying of the resin, carried out as described below:
Deprotection (2500 μl 20%/piperidine in DMF) 8 min×1
Deprotection (2500 μl 20%/piperidine in DMF) 12 min×1
Drying (Solvent extraction with the use of a vacuum pump) 30s×1
Rinsing (2210 μl DMF) 1 min×6
Rinsing (2210 μl EtOH) 1 min×5
Drying (Solvent extraction with the use of a vacuum pump) 300s×1

After completion of the last final cycle of automatic synthesis, the resin columns were removed from the unit and the resin was rinsed again with 15 ml of diethyl ether and left in a vacuum desiccator until the next step of synthesis—the linker attachment.

### Synthesis of a HISNHSHHHDIL Molecule Sequence (KOD 1; SEQ ID NO 1)

For the synthesis of SEQ ID NO 1 peptide, the reaction conditions given in Table 1 below were applied.

TABLE 1

| | | Molar | | Weight/volume | | Quantity of | Quantity of | | |
|---|---|---|---|---|---|---|---|---|---|
| | | Mass | Concentra- | of substance used | | solution | solution | | |
| Function | Name | [g/mol] | tion [mol/l] | for preperation | | used | prepared | | Solvent |
| Activator | TBTU + HOBt | 321.08 + | 0.5 | 19.30 g + 8.12 g | | 112.20 | 120 | | DMF |
| | | 135.12 | | | | | | | |
| Alkali | NMM | 101.15 | 4 | 35.20 | ml | 67.50 | 80 | | DMF |
| Piperidine | 20% PIP | 85.15 | 2.02 | 30 | ml | 120.10 | 150 | | DMF |
| Solvent 1 | NMP | 99.13 | — | 5 | | 2.390 | 5 | | — |
| Solvent 2 | EtOH | 46.07 | — | 50 | ml | 16 | 50 | ml | — |
| Solvent 3 | DMF | 73.09 | — | 700 | ml | 505 | 700 | ml | — |

| Derivative 1 | Fmoc-Asn(Trt)-OH | 596.67 | 0.5 | 2.98 | g | 7.22 | 10 | DMF |
|---|---|---|---|---|---|---|---|---|
| Derivative 2 | Fmoc-Asp(OtBu)- | 411.45 | 0.5 | 2.06 | g | 7.22 | 10 | DMF |
| | OH | | | | | | | |
| Derivative 3 | Fmoc-His(Trt)-OH | 619.71 | 0.5 | 9.30 | g | 28.08 | 30 | DMF |
| Derivative 4 | Fmoc-Ile-OH | 353.41 | 0.5 | 2.65 | g | 12.43 | 15 | DMF |
| Derivative 5 | Fmoc-Leu-OH | 353.41 | 0.5 | 1.77 | g | 7.22 | 10 | DMF |
| Derivative 6 | Fmoc-Ser(tBU)- | 383.44 | 0.5 | 2.88 | g | 12.43 | 15 | DMF |

OH

The remaining peptides were synthesized in a similar way, for example:

### Synthesis of a Molecule of the Sequence HMQSHKTHHSQR (KOD 2, SEQ ID NO 2)

For the synthesis of SEQ ID NO 2 peptide, the reaction conditions given in Table 2 below were applied.

TABLE 2

| | | Molar | | Weight/volume | | Quantity of | Quantity of | | |
|---|---|---|---|---|---|---|---|---|---|
| | | mass | Concentra- | of substance used | | solution | solution | | |
| Function | Name | [g/mol] | tion [mol/l] | for preperation | | used | prepared | | Solvent |
| Activator | TBTU + HOBt | 321.08 + | 0.5 | 19.30 g + 8.12 g | | 120.20 | 120 | | DMF |
| | | 135.12 | | | | | | | |
| Alkali | NMM | 101.15 | 4 | 35.20 | ml | 67.50 | 80 | | DMF |
| Piperidine | 20% PIP | 85.15 | 2.02 | 30 | ml | 120.10 | 150 | | DMF |
| Solvent 1 | NMP | 99.13 | — | 5 | | 2.390 | 5 | | — |
| Solvent 2 | EtOH | 46.07 | — | 50 | ml | 16 | 50 | ml | — |
| Solvent 3 | DMF | 73.09 | — | 700 | ml | 505 | 700 | ml | — |

| Derivative 1 | Fmoc-Arg (Pbf)- | 648.8 | 0.5 | 3.24 | g | 7.22 | 10 | DMF |
|---|---|---|---|---|---|---|---|---|
| | OH | | | | | | | |
| Derivative 2 | Fmoc-Gln(Trt)-OH | 610.7 | 0.5 | 4.59 | g | 12.43 | 15 | DMF |
| Derivative 3 | Fmoc-His(Trt)-OH | 619.71 | 0.5 | 7.75 | g | 22.86 | 25 | DMF |
| Derivative 4 | Fmoe-Lys(Boc)- | 468.5 | 0.5 | 2.34 | g | 7.22 | 10 | DMF |
| | OH | | | | | | | |
| Derivative 5 | Fmoc-Thr(tBu)-OH | 397.5 | 0.5 | 1.99 | g | 7.22 | 10 | DMF |
| Derivative 6 | Fmoc-Ser(tBU)-OH | 383.44 | 0.5 | 2.88 | g | 12.43 | 15 | DMF |
| Derivative 7 | Fmoc-Met-OH | 371.6 | 0.5 | 1.86 | g | 7.22 | 10 | DMF |

### Synthesis of a Molecule of the Sequence FSLPSTL (KOD 5, SEQ ID NO 5)

For the synthesis of SEQ ID NO 5 peptide, the reaction conditions given in Table 3 below were applied.

TABLE 3

| | | Molar | | Weight/volume | | Quantity of | Quantity of | | |
|---|---|---|---|---|---|---|---|---|---|
| | | mass | Concentra- | of substance used | | solution | solution | | |
| Function | Name | [g/mol] | tion [mol/l] | for preperation | | used | prepared | | Solvent |
| Activator | TBTU + HOBt | 321.08 + | 0.5 | 16.10 g + 6.77 g | | 87.40 | 100 | | DMF |
| | | 135.12 | | | | | | | |
| Alkali | NMM | 101.15 | 4 | 30.80 | ml | 61.30 | 70 | | DMF |
| Piperidine | 20% PIP | 85.15 | 2.02 | 20 | ml | 94.20 | 100 | | DMF |
| Solvent 1 | NMP | 99.13 | — | 5 | | 2.390 | 5 | | — |
| Solvent 2 | EtOH | 46.07 | — | 20 | ml | 16 | 20 | ml | — |
| Solvent 3 | DMF | 73.09 | — | 400 | ml | 316 | 400 | ml | — |

| Derivative 1 | Fmoc-Phe-OH | 387.4 | 0.5 | 1.94 | g | 7.22 | 10 | DMF |
|---|---|---|---|---|---|---|---|---|
| Derivative 2 | Fmoc-Pro-OH | 377.4 | 0.5 | 1.69 | g | 7.22 | 10 | DMF |
| Derivative 3 | Fmoc-Thr(tBu)- | 397.5 | 0.5 | 1.99 | g | 7.22 | 10 | DMF |
| | OH | | | | | | | |
| Derivative 4 | Fmoc-Leu-OH | 353.41 | 0.5 | 2.65 | g | 12.43 | 15 | DMF |
| Derivative 5 | Fmoc-Ser(tBu)- | 383.44 | 0.5 | 2.88 | g | 12.43 | 15 | DMF |

OH

The attachment of the derivative to the peptide chain was carried out each time manually or automatically in the following manner.

### Attachment of a Derivative to a Peptide Chain

11-Mercaptooctanoic acid (11-Mrpct) (2 eq relative to the degree of resin deposition) [alternative pathway: 8-Mercaptooctanoic acid (8-Mrpct) or 6-Mercaptohexanoic acid (6-Mrpct)] was dissolved in a small amount of DMF solution, DIC (2 eq) and HOBt (2 eq) were added.

Then it was all vortexed. The prepared solution was drawn into a syringe containing peptidyl resin and placed on a laboratory bench rocker. The acylation reaction was conducted for 45 minutes. Then the solution was removed from the syringe, a fresh portion of the mixture was drawn and the reaction was repeated. At the end of the reaction the solution was removed from the syringe, and peptidyl resin was rinsed successively with DMF (3×), DCM (3×), DMF (3×) solution.

In order to assess the effectiveness of acylation, a chloranilic test was performed (red colouring of the grains indicates the attachment of the derivative)

Attachment of Derivative to Peptide Chain Using Microwave Reactor Magnum Nova 10 MW EARTEC Reactor 800 W—an Alternative Method of Introducing a Derivative into the Peptide Chain.

The peptidyl resin was placed in a synthesis vessel in a microwave reactor and DMF solution was added to swell it. After 30 minutes the solution was removed. 11-Merkaptoundecanoic acid, 11-Mrpct (4 eq towards the degree of resin deposition) was dissolved in a small amount of DMF solution, DIC (4 eq) and HOBt (4 eq) were added, everything was vortexed. The solution of the acyclic mixture prepared in this way was transferred to a vessel containing peptidyl resin. Then the vessel was placed in a microwave reactor. The reaction was carried out for 5 minutes using 7% power and mixing with nitrogen stream. After draining the solution, a fresh portion of the acyclic mixture was introduced into the vessel and the reaction was repeated. The preparation of the acyclic mixture and the conditions of the reaction were identical as described above. At the end of the reaction, the solution was drained and the peptidyl resin was rinsed, consecutively with DMF (3×), DCM (3×), DMF (3×) solution. In order to assess the effectiveness of acylation the chloranilic test was performed. Red colouring of the grains of the resin indicates the attachment of the 11-Mrpct derivative.

The obtained raw bioreceptor molecule with general formula HS—CH₂(CH₂)₈CH₂—CONH-[peptide sequence]-NH₂ and HS—CH₂(CH₂)₈CH₂—CONH-[peptide sequence]-NH₂ were purified by reverse phase high-performance liquid chromatography. For purification a preparation column type C18 was used in linear gradient, where the mobile phase is a system of solvents A and B (A—H₂O+0.1% TFA, B—100% ACN+0.1% TFA). Eluates were fractionated and then analysed with the RP-HPLC analytical method with 0-100% B linear gradient (A—H₂O+0.1% TFA, B—100% ACN+0.1% TFA) on C18 type analytical column (FIG. 1-6). The fractions of the highest purity were combined and lyophilized.

The synthesized and purified compounds were characterized by mass spectrometry. (Table 4, FIGS. 7-11).

TABLE 4

| | | Reten- | Mass | |
|---|---|---|---|---|
| | | tion | Theo- | |
| Molecule | | time | ret- | Ob- |
| name | Sequence | [min] | ical | served |
| 11M-K0D1- | HS- | 12.85 | 1644.7 | 1645.5 |
| NH2 | CH₂(CH₂)₈CH₂C(O)- | | | [M + H]⁺ |
| | HISNHSHHHDIL- | | | |
| | NH2 | | | |
| | (SEQ ID NO 1) | | | |
| 11M-KOD2- | HS- | 12.051 | 1711.1 | 1712.5 |
| NH₂ | CH₂(CH₂)₈CH₂C(O)- | | | [M + H]+ |
| | HMQSHKTHHSQR- | | | |
| | NH₂ | | | |
| | (SEQ ID NO 2) | | | |
| 11M-KOD5- | HS- | 22.322 | 962.4 | 985.3 |
| NH₂ | CH₂(CH₂)₈CH₂C(O)- | | | [M + Na]+ |
| | FSLPSTL-NH₂ | | | |
| | (SEQ ID NO 5) | | | |
| 11M-KOD6- | HS- | 16.813 | 1117.5 | 1118.9 |
| NH2 | CH₂(CH₂)₈CH₂C(O)- | | | [M + H]+ |
| | SFPVTLQK-NH₂ | | | |
| | (SEQ ID NO 6) | | | |
| 11M-K0D7- | HS- | 16.841 | 1069.5 | 1070.4 |
| NH2 | CH₂(CH₂)₈CH₂C(O)- | | | [M + H]+ |
| | TPIYHKL-NH₂ | | | |
| | (SEQ ID NO 7) | | | |
| 8M-KOD5- | HS- | 17.483 | 920.4 | 943.3 |
| NH₂ | CH2(CH₂)₅CH₂ | | | [M + Na]+ |
| | C(O)-)- | | | |
| | FSLPSTL-NH₂ | | | |
| | (SEQ ID NO 5) | | | |
| 8-KOD1- | HS- | 9.744 | 1602.7 | 1603.4 |
| NH2 | CH₂(CH₂)₅CH₂C(O)- | | | [M + H]+ |
| | HISNHSHHHDIL- | | | |
| | NH₂ | | | |
| | (SEQ ID NO 1) | | | |

In compliance with the content of Examples 1 and 2, the particles shown in table 5 below were obtained.

TABLE 5

| MARK- | | |
|---|---|---|
| ING | SEQUENCE | FORMULA |
| 8-M- KOD-1 | 8-Mrcpt- HISNHS- HHHDIL- NH₂ (SEQ ID NO 1) | 8-Mrcpt-HISNHSHHHDIL-NH2 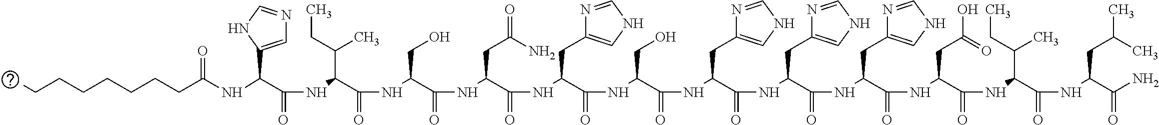 [SMILES: CCCCCCCCC(=O)N[C@H](C(=O)N[C@H](C(=O)N[C@@H](CO)C(=O)N[C@@H](CC(N)=O)C(=O)N[C@@H](Cc1c[nH]cn1)C(=O)N[C@@H](CO)C(=O)N[C@@H](Cc1c[nH]cn1)C(=O)N[C@@H](Cc1c[nH]cn1)C(=O)N[C@@H](Cc1c[nH]cn1)C(=O)N[C@@H](CC(=O)O)C(=O)N[C@H](C(=O)N[C@@H](CC(C)C)C(N)=O)C(C)CC)C(C)CC)c1cnc[nH]1] |
| 11-M- KOD-1 | 11-Mrcpt- HISNHS- HHHDIL- NH₂ (SEQ ID NO 1) | 11-Mrcpt-HISNHSHHHDIL-NH2 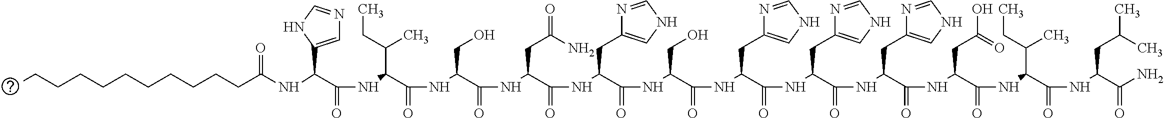 [SMILES: CCCCCCCCCCCC(=O)N[C@H](C(=O)N[C@H](C(=O)N[C@@H](CO)C(=O)N[C@@H](CC(N)=O)C(=O)N[C@@H](Cc1c[nH]cn1)C(=O)N[C@@H](CO)C(=O)N[C@@H](Cc1c[nH]cn1)C(=O)N[C@@H](Cc1c[nH]cn1)C(=O)N[C@@H](Cc1c[nH]cn1)C(=O)N[C@@H](CC(=O)O)C(=O)N[C@H](C(=O)N[C@@H](CC(C)C)C(N)=O)C(C)CC)C(C)CC)c1cnc[nH]1] |
| 8-M- KOD-2 | 8-Mrcpt- HMQSH- KTHHSQR- NH₂ (SEQ ID NO 2) | 8-Mrcpt-HMQSHKTHHSQR-NH2 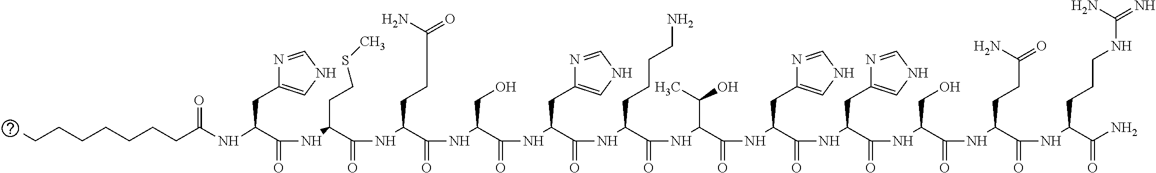 [SMILES: CCCCCCCCC(=O)N[C@@H](Cc1c[nH]cn1)C(=O)N[C@@H](CCSC)C(=O)N[C@@H](CCC(N)=O)C(=O)N[C@@H](CO)C(=O)N[C@@H](Cc1c[nH]cn1)C(=O)N[C@@H](CCCCN)C(=O)NC(C(=O)N[C@@H](Cc1c[nH]cn1)C(=O)N[C@@H](Cc1c[nH]cn1)C(=O)N[C@@H](CO)C(=O)N[C@@H](CCC(N)=O)C(=O)N[C@@H](CCCNC(=N)N)C(N)=O)[C@@H](C)O] |
| 11-M- KOD-2 | 11-Mrcpt- HMQSH- KTHHSQR- NH₂ (SEQ ID NO 2) | 11-Mrcpt-HMQSHKTHHSQR-NH2 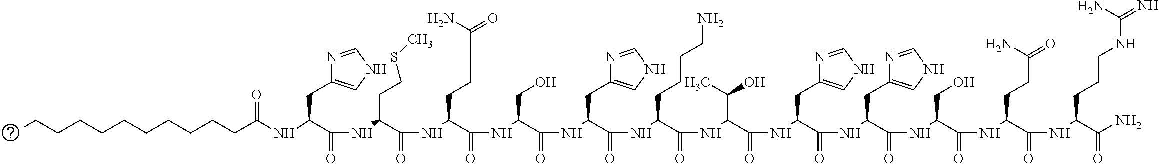 [SMILES: CCCCCCCCCCCC(=O)N[C@@H](Cc1c[nH]cn1)C(=O)N[C@@H](CCSC)C(=O)N[C@@H](CCC(N)=O)C(=O)N[C@@H](CO)C(=O)N[C@@H](Cc1c[nH]cn1)C(=O)N[C@@H](CCCCN)C(=O)NC(C(=O)N[C@@H](Cc1c[nH]cn1)C(=O)N[C@@H](Cc1c[nH]cn1)C(=O)N[C@@H](CO)C(=O)N[C@@H](CCC(N)=O)C(=O)N[C@@H](CCCNC(=N)N)C(N)=O)[C@@H](C)O] |
| 8-M- KOD-3 | 8-Mrcpt- HTVHA- HHASHLS- NH₂ (SEQ ID NO 3) | 8-Mrcpt-HTVHAHHASHLS-NH2 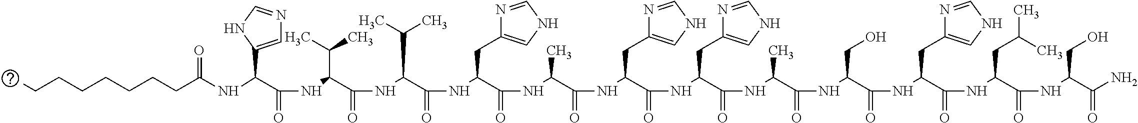 |
| 11-M- KOD-3 | 11-Mrcpt- HTVHA- HHASHLS- NH₂ (SEQ ID NO 3) | 11-Mrcpt-HTVHAHHASHLS-NH2 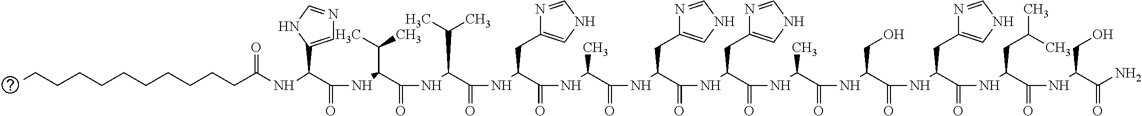 |
| 8-M- KOD-4 | 8-Mrcpt- IWGKS- YHIHSLH- NH₂ (SEQ ID NO 4) | 8-Mrcpt-IWGKSYHIHSLH-NH2 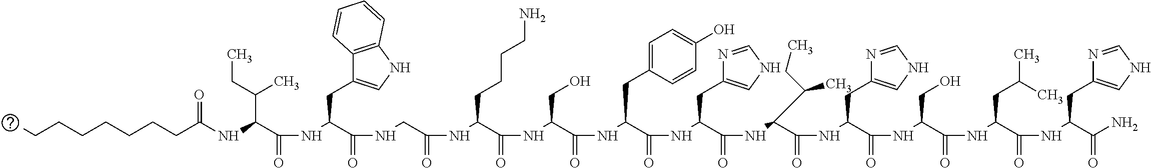 [SMILES: CCCCCCCCC(=O)N[C@H](C(=O)N[C@@H](Cc1c[nH]c2ccccc12)C(=O)NCC(=O)N[C@@H](CCCCN)C(=O)N[C@@H](CO)C(=O)N[C@@H](Cc1ccc(O)cc1)C(=O)N[C@@H](Cc1c[nH]cn1)C(=O)N[C@H](C(=O)N[C@@H](Cc1c[nH]cn1)C(=O)N[C@@H](CO)C(=O)N[C@@H](CC(C)C)C(=O)N[C@@H](Cc1c[nH]cn1)C(N)=O)[C@H](C)CC)C(C)CC] |
| 11-M- KOD-4 | 11-Mrcpt- IWGKS- YHIHSLH- NH₂ (SEQ ID NO 4) | 11-Mrcpt-IWGKSYHIHSLH-NH2 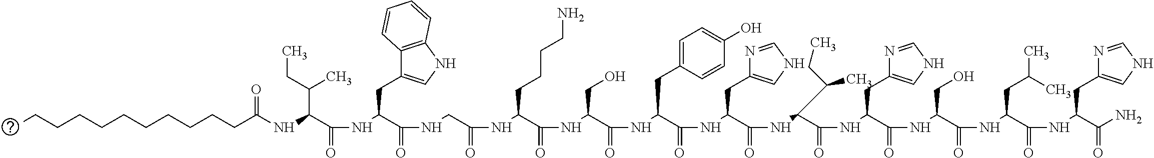 [SMILES: CCCCCCCCCCCC(=O)N[C@H](C(=O)N[C@@H](Cc1c[nH]c2ccccc12)C(=O)NCC(=O)N[C@@H](CCCCN)C(=O)N[C@@H](CO)C(=O)N[C@@H](Cc1ccc(O)cc1)C(=O)N[C@@H](Cc1c[nH]cn1)C(=O)N[C@H](C(=O)N[C@@H](Cc1c[nH]cn1)C(=O)N[C@@H](CO)C(=O)N[C@@H](CC(C)C)C(=O)N[C@@H](Cc1c[nH]cn1)C(N)=O)[C@H](C)CC)C(C)CC] |
| 8-M- KOD-5 | 8-Mrcpt- FSLPSTL- NH₂ (SEQ ID NO 5) | 8-Mrcpt-FSLPSTL-NH2 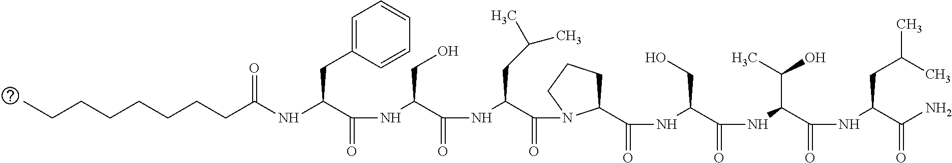 [SMILES: CCCCCCCCC(=O)N[C@@H](Cc1ccccc1)C(=O)N[C@@H](CO)C(=O)N[C@@H](CC(C)C)C(=O)N1CCC[C@H]1C(=O)N[C@@H](CO)C(=O)N[C@H](C(=O)N[C@@H](CC(C)C)C(N)=O)[C@@H](C)O] |
| 11-M- KOD-5 | 11-Mrcpt- FSLPSTL- NH₂ (SEQ ID NO 5) | 11-Mrcpt-FSLPSTL-NH2 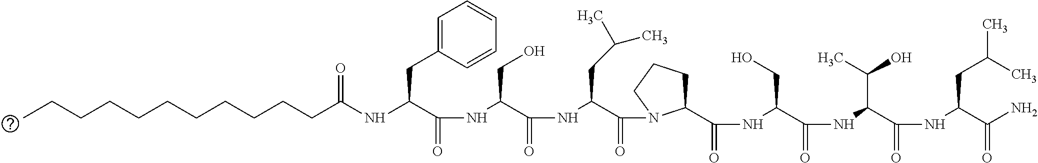 [SMILES: CCCCCCCCCCCC(=O)N[C@@H](Cc1ccccc1)C(=O)N[C@@H](CO)C(=O)N[C@@H](CC(C)C)C(=O)N1CCC[C@H]1C(=O)N[C@@H](CO)C(=O)N[C@H](C(=O)N[C@@H](CC(C)C)C(N)=O)[C@@H](C)O] |
| 8-M- KOD-6 | 8-Mrcpt- SFPVTLQK- NH₂ (SEQ ID NO 6) | 8-Mrcpt-SFPVTLQK-NH2 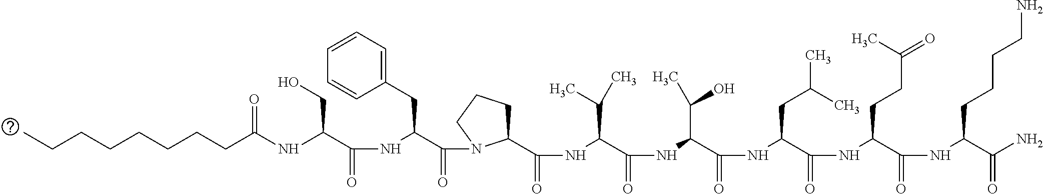 [SMILES: CCCCCCCCC(=O)N[C@@H](CO)C(=O)N[C@@H](Cc1ccccc1)C(=O)N1CCC[C@H]1C(=O)N[C@H](C(=O)N[C@H](C(=O)N[C@@H](CC(C)C)C(=O)N[C@@H](CCC(C)=O)C(=O)N[C@@H](CCCCN)C(N)=O)[C@@H](C)O)C(C)C] |
| 11-M- KOD-6 | 11-Mrcpt- SFPVTLQK- NH₂ (SEQ ID NO 6) | 11-Mrcpt-SFPVTLQK-NH2 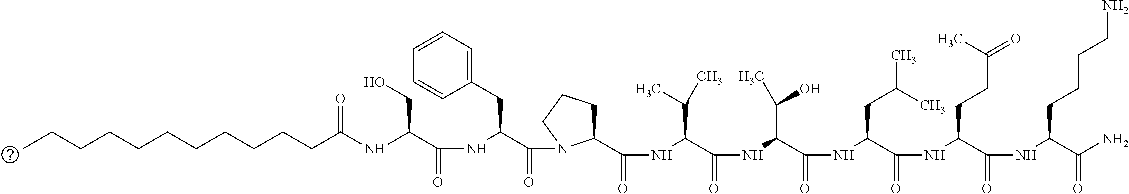 [SMILES: CCCCCCCCCCCC(=O)N[C@@H](CO)C(=O)N[C@@H](Cc1ccccc1)C(=O)N1CCC[C@H]1C(=O)N[C@H](C(=O)N[C@H](C(=O)N[C@@H](CC(C)C)C(=O)N[C@@H](CCC(C)=O)C(=O)N[C@@H](CCCCN)C(N)=O)[C@@H](C)O)C(C)C] |
| 8-M- KOD-7 | 8-Mrcpt- TPIYHKL- NH₂ (SEQ ID NO 7) | 8-Mrcpt-TPIYHKL-NH2 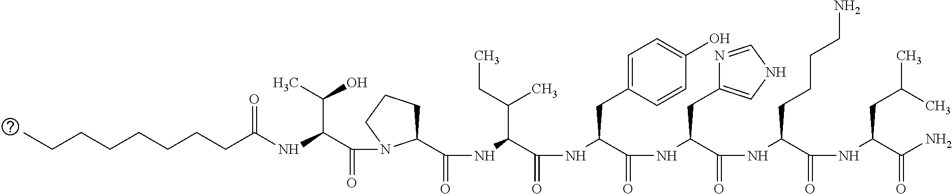 [SMILES: CCCCCCCCC(=O)N[C@H](C(=O)N1CCC[C@H]1C(=O)N[C@H](C(=O)N[C@@H](Cc1ccc(O)cc1)C(=O)N[C@@H](Cc1c[nH]cn1)C(=O)N[C@@H](CCCCN)C(=O)N[C@@H](CC(C)C)C(N)=O)C(C)CC)[C@@H](C)O] |
| 11-M- KOD-7 | 11-Mrcpt- TPIYHKL- NH₂ (SEQ ID NO 7) | 11-Mrcpt-TPIYHKL-NH2 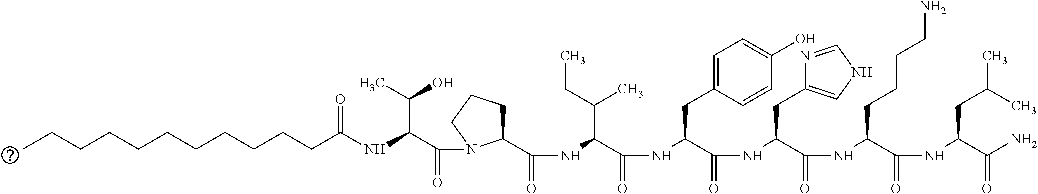 [SMILES: CCCCCCCCCCCC(=O)N[C@H](C(=O)N1CCC[C@H]1C(=O)N[C@H](C(=O)N[C@@H](Cc1ccc(O)cc1)C(=O)N[C@@H](Cc1c[nH]cn1)C(=O)N[C@@H](CCCCN)C(=O)N[C@@H](CC(C)C)C(N)=O)C(C)CC)[C@@H](C)O] |
| 8-M- KOD-8 | 8-Mrcpt- HSMHHRH- NH₂ (SEQ ID NO 8) | 8-Mrcpt-HSMHHRH-NH2 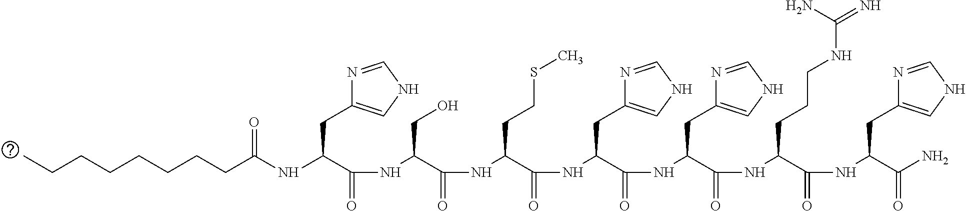 [SMILES: CCCCCCCCC(=O)N[C@@H](Cc1c[nH]cn1)C(=O)N[C@@H](CO)C(=O)N[C@@H](CCSC)C(=O)N[C@@H](Cc1c[nH]cn1)C(=O)N[C@@H](Cc1c[nH]cn1)C(=O)N[C@@H](CCCNC(=N)N)C(=O)N[C@@H](Cc1c[nH]cn1)C(N)=O] |
| 11-M- KOD-8 | 11-Mrcpt- HSMHHRH- NH₂ (SEQ ID NO 8) | 11-Mrcpt-HSMHHRH-NH2 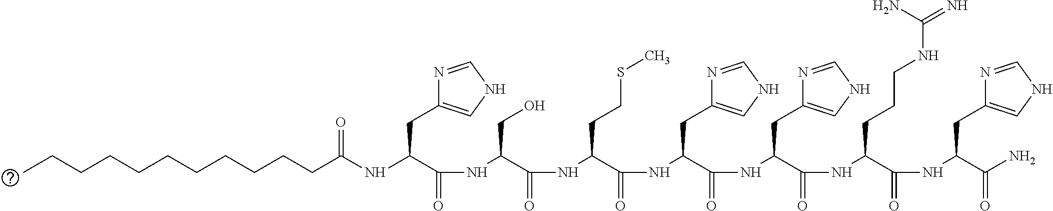 [SMILES: CCCCCCCCCCCC(=O)N[C@@H](Cc1c[nH]cn1)C(=O)N[C@@H](CO)C(=O)N[C@@H](CCSC)C(=O)N[C@@H](Cc1c[nH]cn1)C(=O)N[C@@H](Cc1c[nH]cn1)C(=O)N[C@@H](CCCNC(=N)N)C(=O)N[C@@H](Cc1c[nH]cn1)C(N)=O] |

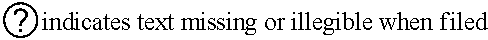

### Example 3

### Cleaning of Gold Electrodes

The gold electrodes on a PCB plate with HDMI output were cleaned before use with NaOH solution and ammonia/hydrogen peroxide mixture diluted with deionized water at a volume ratio of 8:1:1 respectively. The panels with the electrodes were placed in an ultrasonic cleaner and then immersed in 1M NaOH solution for 5 minutes at a temperature above 40° C. After 5 minutes the electrodes were removed from the washing solution and rinsed with deionised water. Then the panel was immersed in the prepared mixture of ammonia with hydrogen peroxide and left for 5 minutes. The electrodes were rinsed with deionized water and then immersed in deionized water for another 5 minutes. The last step of the electrodes washing procedure is to dry them in an argon stream. After this step the electrodes are ready for modifications.

### Example 4

Electrode Surface Modification with 11-KOD5 Bioreceptor Molecules (SEO ID NO 5)

A solution of peptide 11-KOD5 (SEQ ID NO 5) modified with thiol group was applied to the cleaned gold surface. The sequence (FSLPSTL; SEQ ID NO 5) of the peptide (11-KOD5) is specific for SARS-CoV-2 by recognizing the WHN-N protein. The peptide is dissolved in a mixture of acetonitrile and deionized water at a volumetric ratio of 7:13 (ACN:WDI) to a concentration of 5.20·10⁻⁴ M. The resulting peptide solution was diluted with deionized water up to the concentration of 5·10⁻⁵ M. In order to modify, 2.6 μl of the solution containing peptide was applied to the electrode surface and left in a dark place with 100% humidity, 5-6° C. for 22 h. After this time the unbound peptide was rinsed with deionized water and then the electrode surface was dried in an argon stream. The next step is to test the interaction of the sensor with a positive sample (POZ) in the form of SARS-CoV-2 (WHN-N) capside building N protein suspended in TBS buffer, to which the peptide is sensitive, as well as with negative samples (NEG), which do not contain protein, to which peptide 11-KOD5 of FSLPSTL sequence is specific.

### Example 5

### Testing the Presence of WHN-N Protein in the Sample

A modified electrode as described above was used for the experiments. Positive sample (POZ) is a solution of WHN-N protein suspended in TBS buffer. The measurement electrode was placed in HDMI edge connector using a potentiostat containing FRA card for impedance measurements (Autolab M204).

Approximately 150 μl of measurement buffer composed of 100 mM TRIS-HCl, 6.2 mM K₄[Fe(CN)₆]×3H₂O, 6.2 mM K₃[Fe(CN)₆], 2 M HCl up to pH=7.85, sterile Tween 20 was applied on the electrode surface. The first step of measurement has commenced—electrode calibration. 150 μl of measurement buffer was applied to the electrode, then impedance measurement was performed and the impedances of individual fields on the electrode were checked. During this time, 5 μl of WHN-N protein solution was added to 65 μl of measurement buffer. A solution containing WHN-N protein and measurement buffer was mixed and incubated at room temperature for 1 minute. Then 60 μl of such prepared solution was applied to the electrode adding the solution to the measurement buffer. Impedance measurement was initiated.

The result was considered as positive when impedance changes were at least 10% of the absolute value in relation to the baseline value (FIG. 12).

### Negative Controls:

The sensor interaction test on gold medium with negative samples (NEG) in the form of night culture of Haemophilus influenzae, Streptocococcus pyogenes Streptococcus pneumoniae, and RSV viruses, is carried out as follows:

150 μl of measurement buffer was applied to the individual electrodes modified with 11-KOD5 molecule (SEQ ID NO 5), followed by calibration measurements. Then, onto the electrodes, solutions of Haemophilus influenza, Streptocococcus pyogenes Streptococcus pneumonia bacteria, and RSV virus with a titre of 10⁷ CEID50/mL suspended in TBS were applied. Each measurement was carried out on a separate electrode for a single pathogen.

The results are presented in FIGS. 13-16.

### Example 6

Electrode Modification with a 11-KOD1 Bioreceptor Molecule (SEO ID NO 1)

A solution of bioreceptor molecule 11-KOD1 (SEQ ID NO 1) was applied on a cleaned gold surface. The sequence (HISNHSHHHDI; SEQ ID NO 1) is specific for the WHN-N protein (SARS-CoV-2 capside N protein). The peptide is dissolved in a mixture of acetonitrile and deionized water in a volume ratio of 4:5 (ACN:WDI) to a concentration of 7.43·10⁻⁴ M. The resulting peptide solution was diluted with deionized water to the concentration of 5·10⁻⁵ M. In order to modify, 2.6 μl of the solution containing the bioreceptor molecule was applied to the electrode surface and left in a dark place with 100% humidity, temperature 5-6° C. for 22 h. After this time the unbound peptide was rinsed with deionised water and then the electrode surface was dried in an argon stream. The next step was to examine the interaction of the sensor with the positive sample (POZ) in the form of the SARS CoV-2 (WHN-N) capside building protein suspended in TBS buffer, to which the peptide is sensitive, as well as with the negative samples (NEG), which do not contain protein to which peptide 11-KOD1 of SEQ ID NO 1 is specific.

### Example 7

### Testing the Presence of WHN-N Protein in the Sample

An electrode modified as described above was used for the experiments. The positive test (POZ) is a WHN-N protein solution suspended in TBS buffer at 10 μg/ml. The measurement electrode was placed in HDMI edge connector using a potentiostat containing FRA card for impedance measurements (Autolab M204).

Approximately 150 μl of measurement buffer of 100 mM TRIS-HCl, 6.2 mM K₄[Fe(CN)₆]×3H₂O, 6.2 mM K₃[Fe(CN)₆], 0.1% sterile Tween 20. 2M HCl, was added to the surface of the electrode to adjust pH 7.85. The first step of measurement has commenced—electrode calibration. The electrode was covered with 150 μl of measurement buffer, then impedance measurement was performed and impedances of individual fields on the electrode were checked. During this time 5 μl of WHN-N protein suspension was added to 65 μl of measurement buffer. The solution was mixed and incubated at room temperature for 1 minute. Then 60 μl of this solution was applied to the electrode adding the solution to the measurement buffer. Impedance measurement was initiated. The result was considered as positive when impedance changes were at least 10% of the absolute value in relation to the baseline value (FIG. 17).

### Negative Controls:

Sensor interaction on gold base with NEG samples in the form of night culture of Haemophilus influenzae, Streptococcus pyogenes Streptococcus pneumonia bacteria and RSV virus is carried out as follows:

150 μl of measurement buffer was applied to the individual electrodes modified with 11-KOD1 molecule, followed by a calibration measurement. Then solutions of Haemophilus influenzae, Streptococcus pyogenes Streptococcus pneumonia bacteria and RSV virus with a titre of 10⁷ CEID50/mL suspended in TBS were applied on individual electrodes. Each electrode was measured separately for each pathogen.

The results are presented on FIGS. 18-21.

### Example 8

Electrode Surface Modification with 11-KOD7 Bioreceptor Molecules (SEO ID NO 7)

A solution of 11-KOD7 peptide (SEQ ID NO 7) modified with thiol group was applied to the cleaned gold surface. The sequence (TPIYHKL; SEQ ID NO 7) of peptide (11-KOD7) is specific for SARS-CoV-2 virus. The peptide was dissolved in a mixture of acetonitrile and deionized water in a volume ratio of 2:13 (ACN:WDI) to a concentration of 5.98·10⁻⁴ M. The resulting peptide solution was diluted with deionized water to the concentration of 1·10⁻⁵ M. In order to modify, 2.6 μl of the solution containing peptide was applied to the electrode surface and left in a dark place with 100% humidity, 5-6° C. for 22 h. After this time the unbound peptide was rinsed with deionized water and then the electrode surface was dried in an argon stream. The next step is to test the interaction of the sensor with a positive sample (POZ) in the form of SARSCoV-2 (WHN-N) capside building N protein suspended in TBS buffer, to which the peptide is sensitive, as well as with negative samples (NEG), which do not contain protein to which 11-KOD7 peptide (SEQ ID NO 7) is specific.

### Example 9

### Testing the Presence of WHN-N Protein in the Sample

An electrode modified as described above was used for the experiments. Positive sample (POZ) is a solution of WHN-N protein suspended in TBS buffer. The measurement electrode was placed in HDMI edge connector using a potentiostat containing FRA card for impedance measurements (Autolab M204).

Approximately 150 μl of measurement buffer composed of 100 mM TRIS-HCl, 6.2 mM K₄[Fe(CN)₆]×3H₂O, 6.2 mM K₃[Fe(CN)₆], 6 M HCl, 0.1% sterile Tween20 was applied to the electrode surface. The first step of measurement commenced—electrode calibration. 150 μl of measurement buffer was applied onto the electrode, after which the impedance measurement was performed and impedances of individual fields on the electrodes were checked. At that time, 5 μl of WHN-N protein solution was added to 65 μl of measurement buffer. A solution containing WHN-N protein and measurement buffer were mixed and incubated at room temperature for 1 minute. Then 60 μl of the solution was applied to the electrode by adding the solution to the measurement buffer. Impedance measurement was initiated.

The result was considered as positive when impedance changes were at least 10% of the absolute value in relation to the baseline value (FIG. 22).

### Negative Controls:

The sensor interaction test on gold base with NEG samples in the form of night culture of Haemophilus influenzae, Streptococcus pneumoniae, Streptococcus pyogenes bacteria and RSV, EBV viruses is performed as follows:

150 μl of measurement buffer was applied to the individual electrodes modified with 11-KOD7 molecule (SEQ ID NO 7), followed by calibration measurements. Then solutions of Haemophilus influenzae, Streptococcus pneumoniae, Streptococcus pyogenes bacteria as well as RSV and EBV viruses with a titre of 10⁷ CEID50/mL suspended in TBS were applied to the individual electrodes.

Measurements were conducted separately for each pathogen.

The results are presented in FIG. 23.

### Example 10

### Testing for SARS-CoV-2 in Patient Swabs

In order to confirm the sensitivity of the diagnostic test based on selected peptides, the presence of SARS-CoV-2 virus in swabs taken from COVID-19 patients was measured. The presence of the virus in the swab samples was confirmed by the Real-Time PCR molecular method according to the WHO recommendations.

The swab was taken with a swab stick and dissolved in the buffer composed of: 100 mM TRIS-HCl, 6.2 mM K₄[Fe(CN)₆]×3H₂O, 6.2 mM K₃[Fe(CN)₆], 0.1% Sterile Tween 20, 2 M HCl and the pH was brought up to 7.85.

At the same time a single-use sensor (electrode modified with molecule 11-KOD1 of SEQ ID NO 1) and EIS (electrochemical impedance spectrometer) reader MOBI SensDx were prepared. The following instructions were followed: the MOBI SensDx reader was connected to the computer, then the application included in the kit was launched. A single-use sensor was placed in the HDMI socket of the reader. Approximately 200 μl of measurement buffer was applied to the sensor and the measurement was started. After 1 minute, 50 μl of solution was added to the sensor buffer formed by dissolving the swab. The measurement was continued according to the instructions.

After the measurement was finished, the application showed a positive result (+), which indicated the presence of the virus in the sample. Raw data measured by the MOBI SensDx reader (EU trademark EUTMA-018242325, international patent application PCT/IB2019/050935) is illustrated by FIG. 24.

Similarly, an experiment was performed using a negative swab (from a patient not infected with COVID-19, which was confirmed by PCR). The result on the MOBI SensDx reader showed no impedance changes on the sensor, as shown in FIG. 25.

Analogous results were obtained for sensors modified with the remaining molecules. Summary results for positive swabs on sensors modified with KOD1, KOD3, KOD4, KOD5, KOD6 and KOD7 molecules are shown in FIG. 26. The measurement of negative swabs is illustrated in FIG. 27.

The measurement time is very short and is maximum 5 minutes. This is shown in FIG. 28.

The result was considered as positive when the difference in resistance between R_{CTi} and R_{CTr} is more than Δ>10%, which is schematically shown in FIG. 29.

A sensor based on peptides modified with a flexible linker can be used to detect SARS-CoV-2 virus in biological samples such as swabs from the throat, nasopharynx, nose, faeces, urine and blood samples as well as in water and food samples as well as from veterinary samples such as tissue, faeces, urine, swabs taken from different surfaces. The examples show how easy it is to modify the gold surface of the electrodes with the obtained bioreactor molecules—the reaction is one-step. The electrodes obtained by the modifications, were used to recognize the N protein (nucleotocapsyde protein) in the tested samples. The above examples show that sensors containing the electrode are capable of detecting selectively SARS-CoV-2 infection. The effectiveness of the test was previously confirmed by the gold standard applied in this type of diagnostics, i.e. RT-PCR (FIGS. 24-25).

The use of molecules developed in this way in electrochemical impedance spectroscopy allowed to obtain a diagnostic test which is quick and easy to operate, as shown in the above embodiments.

1. The bioreceptor molecule with the following formula:
R₁-alkyl-C(O)NH—R₂,
wherein
alkyl is linear or branched alkyl with 2 to 20 C atoms;
R₁ is selected from the group comprising thiol group (—SH); disulfide bridge; —S(O)-alkyl, where alkyl is linear or branched and contains 1-3 C atoms; thioether, the thioether contains 1-3 C atoms; thioacid; thionyl group;
R₂ is a peptide with a sequence selected from a group comprising SEQ ID NO 1-8.

2. Bioreceptor molecule according to claim 1, wherein R₁ is selected from a group comprising thiol group, disulfide bridge, —S(O)-alkyl, wherein alkyl is linear or branched and contains 1-3 C atoms.

3. The bioreceptor molecule according to claim 2, wherein R₁ is selected from the group comprising thiol group and disulfide bridge.

4. The use of bioreceptor molecules specified in claim 1 in electrochemical impedance spectroscopy for SARS-CoV-2 virus detection.

5. An electrochemical sensor containing an electrode with surface covered with a metal layer, wherein the metal layer modified by bioreceptor molecules defined in claim 1.

6. The electrochemical sensor according to claim 5, wherein the electrode surface is covered with a layer of silver, copper, platinum, chemical, galvanic or evaporated gold.

7. A method of detection of SARS-Cov-2 virus with electrochemical spectroscopy impedance, the method comprising:
a. rinsing and drying the metal-coated sensor electrode,
b. modification of the sensor electrode surface with bioreceptor molecules,
c. calibration of the measurement system,
d. detection of SARS-Cov-2 virus in a sample by means of a measurement system by the observation of impedance changes, characterized in that the surface modification of the sensor electrodes are carried out using bioreceptor molecules specified in claim 1, wherein the presence of the virus in the test sample is indicated by a change in impedance of at least 10% in absolute value against the baseline value.
